(19) United States
(12) Patent Application Publication
(10) Pub. No.: US 2018/0220900 A1
(43) Pub. Date: Aug. 9, 2018

(54) METHOD FOR GIVING A PROMPT BEFORE BLOOD PRESSURE MONITORING AND CORRESPONDING AMBULATORY BLOOD PRESSURE MONITOR

(71) Applicant: XI'AN SHEN MINDRAY MEDICAL ELECTRONICS RESEARCH INSTITUTE CO., LTD., Xi'an (CN)

(72) Inventors: Tao MENG, Xi'an (CN); Mingli QIU, Xi'an (CN); Zhiping WANG, Xi'an (CN)

(21) Appl. No.: 15/906,897

(22) Filed: Feb. 27, 2018

Related U.S. Application Data
(63) Continuation of application No. PCT/CN2015/088327, filed on Aug. 28, 2015.

(51) Int. Cl. A61B 5/0225; A61B 5/0205; A61B 5/11; A61B 5/00

(52) U.S. Cl. CPC A61B 5/0225; A61B 5/0205; A61B 5/1123; A61B 2562/0223; A61B 5/7285; A61B 2560/0223; A61B 2562/0219; A61B 5/486

(57) ABSTRACT

A method is disclosed for notifying a patient before blood pressure monitoring. Also provided is a dynamic blood pressure monitor, which is used for notifying a patient before commencement of a blood pressure measurement.

### TECHNICAL FIELD

The present disclosure relates to the medical field, and in particular to a method for notifying a patient prior to blood pressure monitoring, as well as a corresponding dynamic blood pressure monitor.

### BACKGROUND

Currently in non-invasive blood pressure (NIBP) measurements, a patient is required to keep in a resting state, preferably with an arm at the horizontal position level with the heart. When a conventional dynamic blood pressure monitor performs a predetermined measurement task, a patient does not know when the measurement is to begin. In many cases, the measurement is very sudden for the patient, resulting in the patient neither keeping his or her body calm, nor keeping his or her arm with the cuff level with the heart, resulting in a measurement failures and/or inaccurate measurement results.

### SUMMARY

According to the present disclosure, before performing a predetermined measurement, a dynamic blood pressure monitor notifies the patient audibly or non-audibly that the measurement is about to start, and may further notify the patient to keep in a resting state with his or her arm with the cuff at the same horizontal position with the heart. In this way, the patient may adjust his or her physical state, increasing the accuracy of the blood pressure measurement.

In one embodiment, a patient notification method includes: detecting, when a preset detection time is reached, a current physical state of a patient with a dynamic blood pressure monitor by means of a physical state sensor, wherein the physical state includes a resting state and a non-resting state; determining whether it is necessary to notify the patient to alter his or her physical state according to the physical state of the patient, and notifying the patient in a predetermined manner if it is necessary; starting, when a measurement time is reached, a blood pressure measurement of the dynamic blood pressure monitor, wherein the measurement time is after the detection time.

If it is determined that the physical state of the patient is in a non-resting state, the patient may be notified by one or more of the following: slightly inflating a cuff of the dynamic blood pressure monitor for a period of time, wherein a pressure strength of the slight inflation less than that of an inflation during a normal measurement; delivering a voice announcement to indicate that a blood pressure measurement is about to start; generating a vibration or a flashing light to indicate that a blood pressure measurement is about to start; and generating notification information and sending the notifying information to a portable electronic device of the patient, wherein the portable electronic device includes a smart phone, a smart bracelet, a smart watch, or a pair of smart glasses.

After notifying the patient in the predetermined manner, and after a predetermined period of time, the method may include: continuing to detect the current physical state of the patient by the physical state sensor, and continuing to notify, in the predetermined manner, if the patient is still in the non-resting state.

When notifying by means of slightly inflating the cuff, the method may include continuing to detect the physical state of the patient after one inflation, and slightly inflating the cuff with an adjusted inflation pressure for a period of time if the patient is still in the non-resting state.

The physical state sensor may include one or more of a gyroscope, an accelerometer, and an electronic compass sensor.

In one embodiment, detecting a current physical state of a patient may include: acquiring a motion parameter of the patient by the physical state sensor placed on the body of the patient; calibrating the motion parameter; and determining the physical state of the patient according to physical state data of the patient that is calculated from the calibrated motion parameter using a sensor data algorithm.

The method may further include a step of setting a means and duration for notifying the patient.

In one embodiment, a dynamic blood pressure monitor may include: a physical state acquisition unit for detecting, when a preset detection time is reached, a current physical state of a patient with a dynamic blood pressure monitor by means of a physical state sensor, the physical state including a resting state and a non-resting state; a notifying processing unit for determining whether it is necessary to notify the patient according to the physical state of the patient, and for notifying the patient in a predetermined manner if it is necessary; and a measurement unit for starting, when a measurement time is reached, a blood pressure measurement procedure of the dynamic blood pressure monitor, wherein the measurement time is after the detection time.

The notifying processing unit may further include: a determination unit for determining whether the physical state of the patient is in the non-resting state, according to the information acquired by the physical state acquisition unit;

The notifying processing unit may further comprises at least one of: an inflation notifying unit for slightly inflating a cuff for a period of time when the determination unit determined the physical state of the patient in the non-resting state, wherein a pressure strength of the slight inflation is less than that of an inflation during a normal measurement; a voice notifying unit for notifying the patient by means of a voice announcement to indicate that the blood pressure measurement is about to start when the determination unit determined the physical state of the patient in the non-resting state; a notifying unit for providing other means for notifying the patient, such as a vibration or a flashing light, to indicate that the blood pressure measurement is about to start when the determination unit determined the physical state of the patient in the non-resting state; and a notifying information sending unit for generating notifying information and sending the notifying information to a portable electronic device of the patient if the determination unit determined the physical state of the patient in the non-resting state, wherein the portable electronic device includes a smart phone, a smart bracelet, a smart watch, or a pair of smart glasses.

In one embodiment, the inflation notifying unit further includes an inflation pressure adjustment unit for adjusting an inflation pressure of current inflation notifying when the patient is detected to be still in the non-resting state after notifying.

The dynamic blood pressure monitor may further include a setting unit for setting the means and duration for notifying the patient.

The embodiments of the present disclosure may result in a number of beneficial effects. Whether a patient is in a non-resting state may be detected by a physical state sensor at a predetermined time before the start of a blood pressure measurement. If it is detected that the patient is in a non-resting state, the patient may be notified by means of slightly inflating or by other ways to indicate that the measurement is to about to start. When the patient receives the notification, the patient may stop any ongoing activity, and maintain his or her arm with a cuff at the same horizontal position with the heart (in a resting state), increasing the accuracy of blood pressure monitoring. Moreover, the patient may select different methodologies for notifying according to different application scenarios, which is quite convenient and practical.

### BRIEF DESCRIPTION OF THE DRAWINGS

FIG. 1 is a schematic diagram of a method for notifying a patient before blood pressure monitoring;

FIG. 2 is a schematic diagram of a method for notifying a patient before blood pressure monitoring;

FIG. 3 is a schematic diagram of a method for notifying a patient before blood pressure monitoring;

FIG. 4 is a schematic diagram of some of the functional structures of a dynamic blood pressure monitor;

FIG. 5 is a schematic structural diagram of a notifying processing unit in FIG. 4; and

FIG. 6 is a schematic structural diagram of an inflation notifying unit in FIG. 5.

### DETAILED DESCRIPTION

The technical solutions of the embodiments of the present disclosure will be described below clearly and comprehensively in conjunction with the. The embodiments described are merely some embodiments of the present disclosure and are not all the possible embodiments. Based on the embodiments given in the present disclosure, all other embodiments that would be obtained by those of ordinary skill in the art without expending inventive effort shall all fall within the scope of protection of the present disclosure.

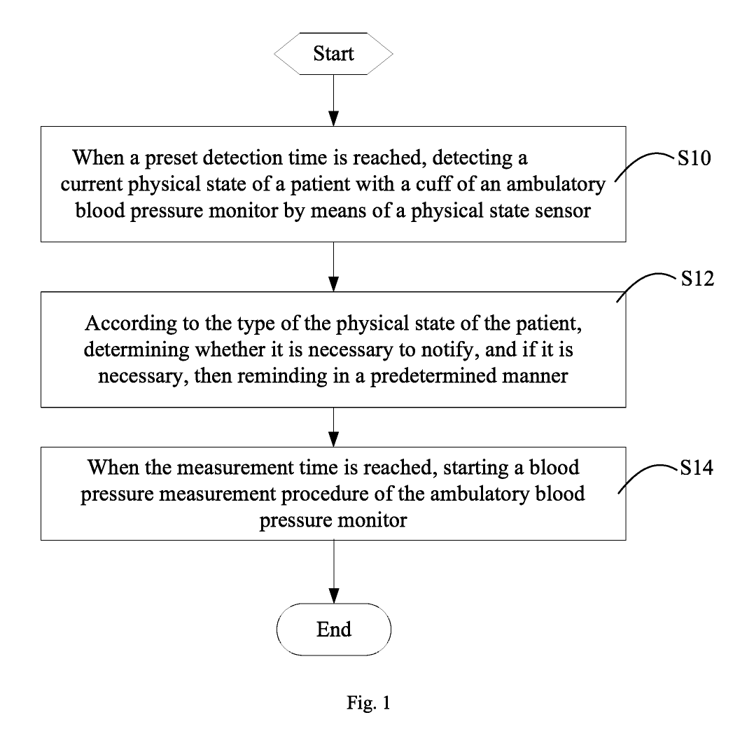

As shown in FIG. 1, a flow chart of an embodiment of a method for notifying a patient before blood pressure monitoring provided by the present disclosure is shown. The method may include the following steps.

In Step S10, when a preset detection time is reached, the method may detect a current physical state of a patient with a dynamic blood pressure monitor by means of a physical state sensor.

The physical state sensor may be configured as a high-performance three-dimensional motion physical state measurement system based on the MEMS (Micro Electro Mechanical Systems) technology. On some occasions, the physical state sensor may be also referred to as a posture sensor. Specifically, the step of detecting the physical state of the patient by means of the physical state sensor may include:

acquiring a motion parameter of the patient by one or more of a gyroscope, an accelerometer, an electronic compass sensor and other motion sensors, placed on the body of the patient (such as an arm or a foot);

calibrating the motion parameter; and

calculating the motion physical state from the calibrated motion parameter using a sensor data algorithm to obtain physical state data of the patient so as to determine the physical state of the patient.

In some embodiments, the motion parameter of the patient may be acquired by one or more of a three-axis gyro, a three-axis accelerometer, and a three-axis electronic compass sensor meter placed on the body of the patient.

The motion parameter may then be calibrated with an embedded low-power ARM processor to obtain calibrated information, such as angular velocity, acceleration, and magnetic data etc.

Thereafter, a motion physical state calculation may be performed on the above information using a quaternion-based sensor data algorithm to obtain in real time three-dimensional physical state data of the patient expressed in quaternion, Euler angle, and the like, and the type of the current physical state of the patient may be determined according to the above three-dimensional physical state data.

Where less accuracy is required, one or more of a biaxial gyroscope, a biaxial accelerometer, and a biaxial electronic compass sensor may be used.

The physical state of the patient obtained in step S10 may include, but are not limited to, a standing state, a lying state, a mild exercise state (such as slight arm swing), a moderate exercise state (such as walking), a strenuous exercise state (such as running) and the like. For ease of description, the physical states hereinafter are divided into resting states and non-resting states. A resting state refers to a state that does not affect blood pressure measurement; and the non-resting state refers to a state that affects blood pressure measurement and may include arm swing, mild exercise, moderate exercise, strenuous exercise, walking, running, and the like.

In Step S12, according to the physical state of the patient, the method may determine whether it is necessary to notify a patient, and if it is necessary, then notifying the patient in a predetermined manner.

In Step S14, when the measurement time is reached, the method may start a blood pressure measurement procedure of the dynamic blood pressure monitor, wherein the measurement time is after the detection time. In one example, there is a preset time difference between the measurement time and the detection time, which may be, for example, tens of seconds to several minutes; or alternatively, in another example, the measurement time may be after a preset period of time since the physical state is detected, that is, the blood pressure measurement procedure is started after a predetermined period of time (such as 10 seconds) since the physical state of the patient is detected. It is to be understood that the preset period of time mentioned above may also be adjusted according to actual needs.

In step S12, if it is determined that the physical state of the patient is a non-resting state, a cuff may be slightly inflated for a period of time, wherein a pressure strength of the slight inflation less than the pressure strength of an inflation during a normal measurement. The pressure strength of the slight inflation may be determined experimentally to ensure that the arm of the wearer (the patient) feels slightly compressed so as to achieve the purpose of the notification. For example, the pressure of the slight inflation may be set to 15 mmHg and the duration may be set to 3 seconds.

In other embodiments, step S12 may further include: after one notification by means of inflating, continuing to detect the physical state of the patient, and if the patient is detected to be still in a non-resting state, slightly inflating the cuff with an adjusted inflation pressure for a period of time. For example, after a current notification, the physical state sensor performs further detection (for example, performs a detection after 2 seconds), and if the patient is detected to be still in a non-resting state, the dynamic blood pressure monitor controls the cuff to be deflated, and then the cuff is inflated with a larger inflation pressure (for example, with an increase of 10 mmHg) for a period of time (such as 3 seconds), so as to achieve the purpose of a better notifying.

Similarly, in addition to the methodologies of slight inflation, a prompt may be given in other methodologies, such as a voice or a non-voice announcement. For example, one or more of the following methodologies for notifying may be used.

In step S12, if it is determined that the physical state of the patient is in the non-resting state, the method may notify the patient by means of the voice announcement to indicate that a blood pressure measurement is to about to start.

It is to be understood that, for each non-resting state, a corresponding voice notification and a corresponding measurement strategy may be correspondingly used. For example, in one embodiment, the following voice notifying methodologies may be used:
a) if it is detected that the patient is in the standing state, notifying the patient as follows: “Please stay still and place your arm at the same level as your heart. The measurement is to about to start”;
b) if it is detected that the patient is in the mild exercise state or in the moderate exercise state, notifying the patient as follows: “Please calm down, stay still, and place the arm at the same horizontal position with the heart. The measurement is about to start”; and
c) if it is detected that the patient is in the strenuous exercise state, notifying the patient as follows: “Please remain in a resting state, and continuing to detect 5 minutes later”.

It is to be understood that in other embodiments, the non-resting states may also not be specifically distinguished, and only a simple voice notification is given, that is, as long as the patient is detected to be in a non-resting state, a fixed voice notification is given, and the voice notification may be, for example, “The measurement is to about to start, please remain quiet.”

In addition, it is to be understood that, in some embodiments, even if the patient is detected to be in a resting state, a corresponding notification may also be given. For example, a notification may be given to the patient to place the arm at the same horizontal position with the heart.

In step S12, if it is determined that the physical state of the patient is in the non-resting state, the method may notify the patient by means of a vibration or a flashing light to indicate that a blood pressure measurement is to about to start.

In step S12, if it is determined that the physical state of the patient is in the non-resting state, the method may generate notifying information and sending the notifying information to a portable electronic device of the patient, wherein the portable electronic device includes a smart phone, a smart bracelet, a smart watch, or a pair of smart glasses.

The method may further comprise a step of setting the means for notifying (such as whether the notification may be a voice notification or a non-voice notification, and whether the notification may be a complex or a simple voice notification) and the duration for notifying.

In step S12, the patient may be notified multiple times. For example, after the first notification is given in the predetermined manner, after a predetermined period of time (such as 2 seconds), the method may continue to detect the current physical state of the patient by the physical state sensor, and if the result of detection is still a non-resting state, then the method may continue to notify in a predetermined manner.

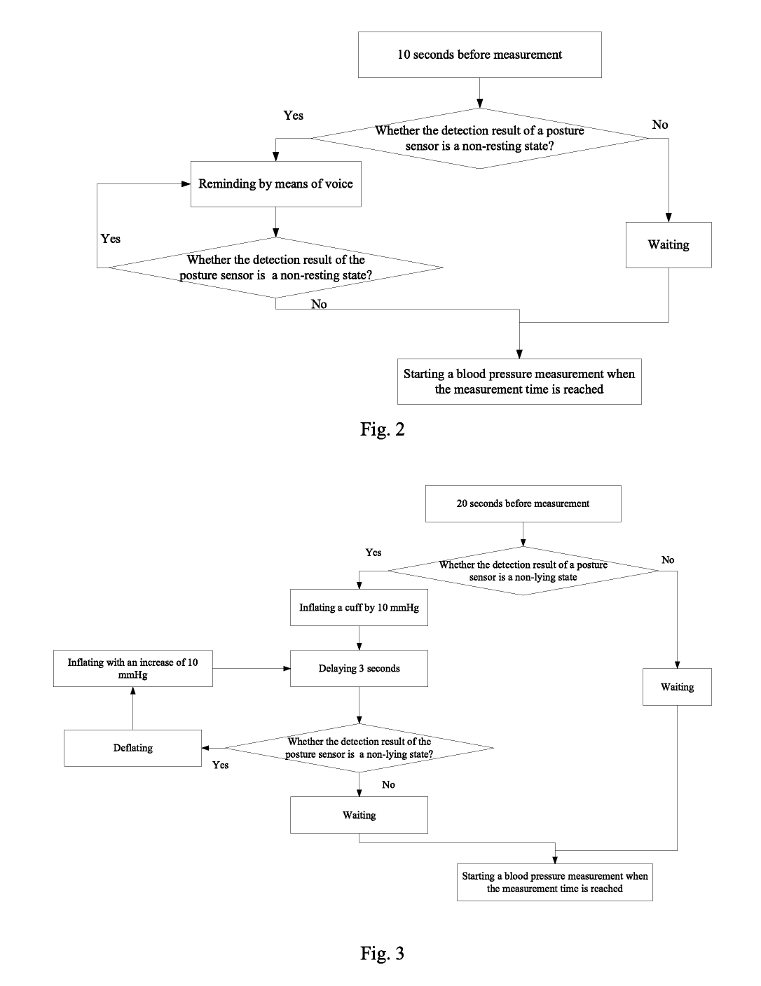

As shown in FIG. 2, an embodiment of a method for notifying a patient before blood pressure monitoring is shown, which includes notifying by means of voice. In this embodiment, a fixed time difference between the measurement time and the detection time may be defined as 10 seconds, that is, a physical state sensor is started for detection 10 seconds before the measurement time is reached. The physical state sensor is generally disposed on a cuff of a dynamic blood pressure monitor, so that the type of a current physical state of the patient with the cuff may be detected by the physical state sensor, the types of physical state includes the resting states and the non-resting states. If the detection result is that the patient is in a resting state, wait for the arrival of the measurement time; and if the detection result is that the patient is in a non-resting state (such as in a motion state), a notifying processing device in the dynamic blood pressure monitor will notify by means of voice, for example, by playing a pre-recorded voice, such as “The blood pressure measurement is to about to start. Please stay still.” After a current voice notification, the physical state sensor will perform a further detection after a predetermined period of time (such as 2 seconds). If the patient is detected to be still in the non-resting state, a voice notification will be given; and if it is detected in the current detection that the patient is in the resting state, the method may wait for the arrival of the measurement time. When the measurement time is reached, a measurement procedure in the dynamic blood pressure monitor is started to begin blood pressure measurement. The manner for notifying is not limited to the voice announcement and other means for notifying may be also used.

The measurement time may be periodic, for example, by detecting once every hour, and the time difference between the measurement time and the previous detection time may be set according to actual conditions. For example, in other embodiments, the time difference may be set to 20 seconds, that is, the detection for the physical state of the patient is started 20 seconds before the measurement time is reached. In addition, the number or manner for voice notification may also be different. For example, when the patient is detected to be in the non-resting state, a fixed number (such as 3 times) of voice notifying is continuously given, and then the blood pressure measurement is started after the measurement time is reached.

FIG. 3 shows an embodiment of a method for notifying a patient before blood pressure monitoring. This manner of notifying may be adopted when a patient equipped with a dynamic blood pressure meter is in a normal working, studying or living condition and does not want to disturb the current environment or when the patient is at a meeting. In this embodiment, a preset time difference between the measurement time and the detection time may be defined as 20 seconds, that is, a physical state sensor is started for detection 20 seconds before the measurement time is reached so as to detect the type of a current physical state of the patient with a cuff, the types of physical state includes a resting state and a non-resting state. If the detection result is that the patient is in the resting state, then the method waits for the arrival of the measurement time; if the detection result is that the patient is in the non-resting state (such as in a motion state), the dynamic blood pressure monitor slightly inflates the cuff via an air pump (for example, with an inflation pressure of 15 mmHg) for a period of time (such as 3 seconds), so that the arm of the patient feels slightly compressed so as to notify the patient to stay in a correct posture, achieving the purpose of notifying before measurement; after the current notification, the physical state sensor performs further detection (for example, performs detection after 2 seconds), and if the patient is detected to be still in the non-resting state, the dynamic blood pressure monitor controls the cuff to be deflated, and then the cuff is inflated with a larger inflation pressure (for example, with an increase of 10 mmHg) for a period of time (such as 3 seconds); if it is detected in the current detection that the patient is in the resting state, then the method waits for the arrival of the measurement time; and when the measurement time is reached, a measurement procedure in the dynamic blood pressure monitor is started to begin blood pressure measurement.

The preset time difference between the measurement time and the previous detection time may be adjusted according to actual conditions; and in addition, the number or manner of inflation notifying may also be different. For example, when it is detected that the patient is in the non-resting state, continuous inflation and deflation may be performed, e.g., for four cycles, and then the blood pressure measurement is started after the measurement time is reached.

In addition, only slight inflation is provided as an example of non-voice notifications, and in other embodiments, a notification may be given in other methodologies, for example, notifying the patient by a vibration or a flashing light, or generating notifying information and sending the notifying information to a portable electronic device of the patient, the portable electronic device includes a smart phone, a smart bracelet, a smart watch, or a pair of smart glasses. For these notification methodologies, corresponding functional modules may be added to the dynamic blood pressure monitor. For example, a vibration device may be added for the manner for notifying by a vibration. An LED light control device may be added for the manner for notifying by a flashing light. A wireless communication device, for example, may be added for the manner for sending notification messages, etc.

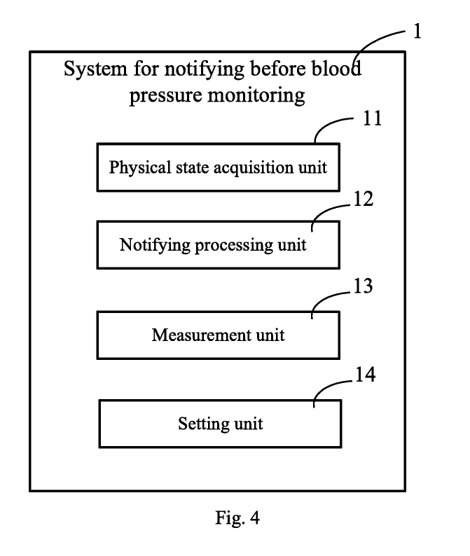

FIG. 4 shows a schematic diagram of an embodiment of a dynamic blood pressure monitor. In this embodiment, the dynamic blood pressure monitor 1 is used for notifying a user before performing blood pressure monitoring on the user, and comprises at least:

a physical state acquisition unit 11 for detecting, when a preset detection period is reached, a current physical state of a patient equipped with a cuff of the dynamic blood pressure monitor by means of a physical state sensor; the physical state may include a resting state and a non-resting state, wherein the non-resting state may include at least one of standing, mild exercise, moderate exercise, and strenuous exercise;

a notifying processing unit 12 for determining whether it is necessary to notify according the physical state of the patient, and for notifying in a predetermined manner if it is necessary; and

a measurement unit 13 for starting, when a measurement time is reached, a blood pressure measurement procedure of the dynamic blood pressure monitor, wherein the measurement time is after the detection time. For example, there is a preset time difference between the measurement time and the detection time, which may be, for example, tens of seconds to several minutes; or alternatively, in another example, the measurement time may be after a preset period of time since the physical state is detected, that is, the blood pressure measurement procedure is started after the predetermined period of time (such as 10 seconds) since the physical state of the patient is detected. The preset period of time mentioned above may also be adjusted according to actual needs.

A setting unit 14 for setting means and duration for notifying, for example, in some scenarios, the means for notifying is set to notify only by slight inflation, and in other scenarios, the means for notifying is set to notify by both a slight inflation and a voice announcement, etc.

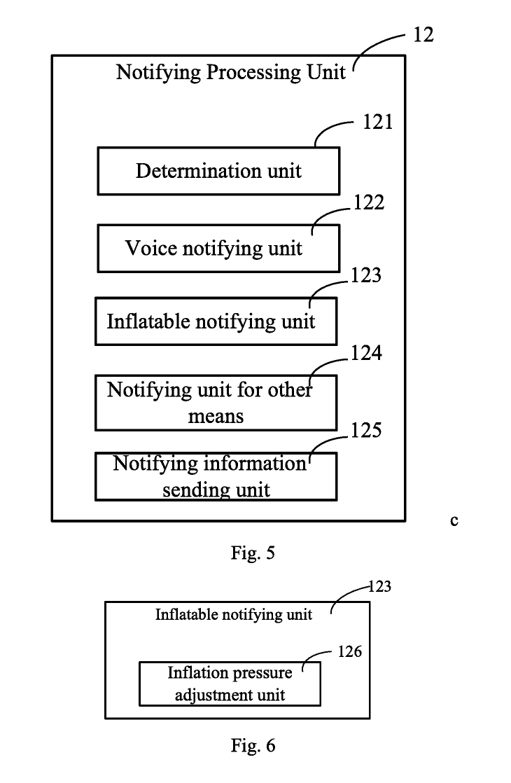

FIG. 5 is a schematic structural diagram of the notifying processing unit in FIG. 4. The notifying processing unit 12 may further include:

a determination unit 121 for determining whether the physical state of the patient to be in the non-resting state, according to the information acquired by the physical state acquisition unit, which is implemented as described above;

a voice notifying unit 122 for notifying the patient by means of a voice announcement to indicate that a blood pressure measurement is to about to start if the determination unit determined the physical state of the patient to be in the non-resting state;

an inflation notifying unit for slightly inflating a cuff for a period of time if the determination unit determines that the physical state of the patient is a non-resting state, wherein a pressure strength of the slight inflation less than the pressure strength of an inflation during normal measurement;

a notifying unit for other means 124 for notifying the patient by means of a vibration or a flashing light to indicate that a blood pressure measurement is about to start, if the determination unit determined the physical state of the patient in the non-resting state; and

a notifying information sending unit 125 for generating notifying information and sending the notifying information to a portable electronic device of the patient, if the determination unit determines that the physical state of the patient is in a non-resting state, wherein the portable electronic device includes a smart phone, a smart bracelet, a smart watch, or a pair of smart glasses.

In the notifying processing unit 12, the voice notifying unit 122, the inflation notifying unit 123, the notifying unit for other means 124, and the notifying information sending unit 125 need not be separate units or included in every embodiment. In some embodiments, the notifying processing unit 12 comprises the determination unit 121 and one or more of the voice notifying unit 122, the inflation notifying unit 123, the notifying unit for other means 124, and the notifying information sending unit 125.

Further, the notifying processing unit 12 may, after a predetermined period of time since notifying in the predetermined manner, continue to detect the current physical state of the patient by a physical state acquisition unit 11, and if the patient is detected to be still in a non-resting state, continue to notify in the predetermined manner.

FIG. 6 is a schematic structural diagram of an embodiment of the inflation notifying unit in FIG. 5. In this embodiment, the inflation notifying unit 123 further comprises:

an inflation pressure adjustment unit 126 for, after one notifiation by means of inflating, if the patient is detected to be still in a non-resting state, adjusting an inflation pressure of current inflation. The dynamic blood pressure monitor imay comprise commonly used functional modules in an existing dynamic blood pressure monitor, for example, a central processing unit, a cuff, a sensor, an A/D converter, an air pump, an air valve, and a display unit and an input unit (not shown in the figure); and in addition, a physical state sensor is provided on the cuff, and a voice play device, a vibration device, a wireless communication device, and/or other functional devices may be added as needed.

In summary, by implementing the embodiments of the present disclosure, the following beneficial effects may be achieved. Whether a patient is in a non-resting state may be detected by a physical state sensor at a predetermined time before the start of a blood pressure measurement, and if it is detected that the patient is in a non-resting state, the patient may be notified by a slight inflation or by other means to indicate that the measurement is about to start, and when the patient receives the notification, the patient may stop the ongoing activity, and maintain an arm with a cuff at the same horizontal position with an heart (in a resting state), so that a result measured by the dynamic blood pressure monitor is more accurate, and the measurement result may better reflect a real blood pressure condition of the patient.

Moreover, the patient may select different methodologies for notifying according to different application scenarios, which is quite convenient and practical.

It is to be understood that those of ordinary skill in the art would be able to understand that implementation of all or some of the procedure of the exemplary methods described above could be achieved by hardware commanded by a computer program, which program may be stored in a computer-readable storage medium and when executed, carry out a procedure as shown in the embodiments of the methods described above. The storage medium may be a magnetic disk, an optical disc, a read-only memory (ROM), a random access memory (RAM), etc.

The technical features or operation steps described in the embodiments of the present application may be combined in any suitable manner. Those skilled in the art may easily understand that the order of the steps or actions in the method described in the embodiments of the present disclosure may be changed.

Therefore, unless a certain sequence is specified otherwise, any sequence in the figures or the detailed description is merely for the purpose of illustration and not an obligatory sequence.

The disclosure above is merely a preferred embodiment of the present application and certainly cannot be used to limit the scope of the present disclosure. Therefore, equivalent changes made according to the claims of the present application are still covered by the present disclosure.

What is claimed is:

1-11. (canceled)

12. A method for notifying a patient before blood pressure monitoring, comprising:
when a preset detection time is reached, detecting a current physical state of a patient with a dynamic blood pressure monitor using a physical state sensor, wherein the physical state includes a resting state and a non-resting state;
determining whether it is necessary to notify the patient according to the physical state of the patient, and notifying the patient in a predetermined manner if it is necessary;
when a measurement time is reached, starting a blood pressure measurement of the dynamic blood pressure monitor, wherein the measurement time is after the detection time.

13. The method of claim 12, wherein determining comprises:
if the physical state of the patient is determined to be in the non-resting state, notifying the patient by slightly inflating a cuff of the dynamic blood pressure monitor for a period of time, wherein a pressure strength of the slight inflation is less than that of an inflation during a normal measurement.

14. The method of claim 13, further comprising:
when notifying the patient by slightly inflating the cuff, continuing to detect the physical state of the patient after one notification by inflation, and slightly inflating the cuff with an adjusted inflation pressure for a period of time if the physical state of the patient is still in the non-resting state.

15. The method of claim 12, wherein determining comprises:
if the physical state of the patient is determined to be in the non-resting state, notifying the patient using a voice announcement to indicate that the blood pressure measurement is about to start.

16. The method of claim 12, wherein determining comprises:
if the physical state of the patient is determined to be in the non-resting state, notifying the patient using at least one of a vibration or a flashing light to indicate that the blood pressure measurement is about to start.

17. The method of claim 12, wherein determining comprises:
if the physical state of the patient is determined to be in the non-resting state, generating notifying information and sending the notifying information to a portable electronic device of the patient, wherein the portable electronic device is at least one of a smart phone, a smart bracelet, a smart watch, or a pair of smart glasses.

18. The method of claim 12, wherein determining comprises:
after notifying the patient in the predetermined manner, and after a predetermined period of time, continuing to detect the current physical of the patient by the physical state sensor, and continuing to notify in the predetermined manner if the physical state of the patient is still in the non-resting state.

19. The method of claim 12, wherein the physical state sensor includes at least one of a gyroscope, an accelerometer, or an electronic compass sensor.

20. The method of claim 12, wherein detecting comprises:
acquiring a motion parameter of the patient by the physical state sensor placed on the patient;
calibrating the motion parameter; and
determining the physical state of the patient according to physical state data of the patient that is calculated from the calibrated motion parameter using a sensor data algorithm.

21. The method of claim 12, further comprising:
setting at least one of a means or a duration for notifying the patient.

22. A dynamic blood pressure monitor, comprising:
a physical state acquisition unit to detect, when a preset detection time is reached, a current physical state of a patient with a dynamic blood pressure monitor using a physical state sensor, wherein the type of the physical state includes a resting state and a non-resting state;
a notification unit for determining whether it is necessary to notify the patient according to the physical state of the patient, and for notifying the patient in a predetermined manner if it is necessary; and
a measurement unit for starting, when a measurement time is reached, a blood pressure measurement procedure of the dynamic blood pressure monitor, wherein the measurement time is after the detection time.

23. The dynamic blood pressure monitor of claim 22, wherein the notification unit comprises:
a determination unit for determining whether the physical state of the patient is in the non-resting state according to information acquired by the physical state acquisition unit; and
an inflation notification unit for slightly inflating a cuff of the dynamic blood pressure monitor for a period of time if the determination unit determined the physical state of the patient is in the non-resting state, wherein a pressure strength of the slight inflation is less than that of an inflation during a normal measurement.

24. The dynamic blood pressure monitor of claim 23, wherein the inflation notification unit further comprises:
an inflation pressure adjustment unit for adjusting an inflation pressure of a current notification by inflation if the patient is detected to be still in the non-resting state after one notification by inflation.

25. The dynamic blood pressure monitor of claim 22, wherein the notification unit comprises:
a determination unit for determining whether the physical state of the patient is in the non-resting state according to information acquired by the physical state acquisition unit; and
a voice notification unit for notifying the patient using a voice announcement to indicate that the blood pressure measurement is about to start if the determination unit determined the physical state of the patient is in the non-resting state.

26. The dynamic blood pressure monitor of claim 22, wherein the notification unit comprises:
a determination unit for determining whether the physical state of the patient is in the non-resting state according to information acquired by the physical state acquisition unit; and
a notification subunit for notifying the patient using at least one of a vibration or a flashing light to indicate that the blood pressure measurement is about to start if the determination unit determined the physical state of the patient is in the non-resting state.

27. The dynamic blood pressure monitor of claim 22, wherein the notification unit comprises:
a determination unit for determining whether the physical state of the patient is in the non-resting state according to information acquired by the physical state acquisition unit; and
a notification information transmission unit for generating notifying information and sending the notifying information to a portable electronic device of the patient if the determination unit determined the physical state of the patient is in the non-resting state, wherein the portable electronic device includes one or more of a smart phone, a smart bracelet, a smart watch, or a pair of smart glasses.

28. The dynamic blood pressure monitor of claim 22, wherein the notification unit is to continue, after notifying in the predetermined manner and after a predetermined period of time, to detect the current physical of the patient by the physical state sensor, and continue to notify in the predetermined manner if the type of the physical state of the patient is still in the non-resting state.

29. The dynamic blood pressure monitor of claim 22, wherein the physical state acquisition unit is to:
acquire, when a preset detection time is reached, a motion parameter of the patient by the physical state sensor placed on the patient;
calibrate the motion parameter; and
determine the physical state of the patient according to physical state data of the patient that is calculated from the calibrated motion parameter using a sensor data algorithm.

30. The dynamic blood pressure monitor of claim 22, wherein the physical state sensor includes at least one of a gyroscope, an accelerometer, or an electronic compass sensor.

31. The dynamic blood pressure monitor of claim 22, further comprising:
a setting unit for setting at least one of a means or duration for notifying the patient.
